(19) United States
(12) Patent Application Publication
(10) Pub. No.: US 2026/0364301 A1
(43) Pub. Date: Nov. 19, 2026

(54) ONLINE MULTIFACETED THERAPY DELIVERY SYSTEM AND PROCESS THEREOF

(71) Applicant: Phillip Condreay, Huntington, NY (US)

(72) Inventor: Phillip Condreay, Huntington, NY (US)

(21) Appl. No.: 19/442,937

(22) Filed: Jan. 7, 2026

Related U.S. Application Data
(60) Provisional application No. 63/743,105, filed on Jan. 8, 2025.

(51) Int. Cl. A61M 21/00; G06F 3/01; G06F 3/04883; G06F 3/0489; G16H 70/20

(52) U.S. Cl. CPC A61M 21/00; G06F 3/013; G06F 3/04883; G06F 3/0489; G16H 70/20; A61M 2021/0027; A61M 2021/005; A61M 2205/3584

(57) ABSTRACT

The present disclosure relates to an online multifaceted therapy delivery system (100) and process (200) thereof, comprising a user device (102) configured to receive experiential therapy content, display interactive activities, capture user responses, and transmit audiovisual information. The user device (102) includes a display unit (104), a microphone (106), an image capturing unit (108), a speaker unit (110), a touch interface (112), a keyboard (114), and an eye tracking sensor (116). An application server (118) communicates with a database (120) and a communication network (122) to coordinate audiovisual streaming, allocate experiential therapy modules, and maintain synchronization. A processing unit (124) connected through the communication network (122) comprises a session management module (126), a synchronization control module (128), a media stimulus delivery module (130), and a data logging module (132). A user interface (134) presents counselor-driven experiential instructions and transmits real time user interaction information to the processing unit (124).

### FIELD OF THE INVENTION

Embodiments of the present invention relate to field of digital platforms for remote therapeutic interactions and support, and specifically relates to an online multifaceted therapy delivery system and process thereof.

### Description of the Related Art

This invention is enhancing the quality of remote therapy experiences by supporting deeper emotional engagement during online sessions. It is consistently improving the effectiveness of therapeutic communication across geographical barriers. It is allowing clients and counselors to remain immersed in structured therapeutic flow without disruption. It is advancing the acceptability of remote therapy among diverse age groups. It is strengthening rapport building between counselors and clients in remote settings. It is creating a more supportive environment for self-expression and emotional processing throughout therapy.

This invention is increasing personalization in remote therapeutic care by adapting to unique clinical needs. It is continuously supporting the continuity of therapy regardless of the client's physical location. It is enhancing therapist efficiency through streamlined interaction management throughout the session. It is improving engagement and participation of clients who struggle in traditional talk-only therapy formats. It is promoting interactive involvement that sustains attention and reduces disengagement during therapy. It is facilitating better long-term therapeutic outcomes through structured and consistent therapeutic workflows.

This invention is supporting counselors in delivering diverse therapy styles through a single integrated digital environment. It is improving accessibility for clients who face challenges reaching physical clinics. It is enabling uninterrupted collaboration between multiple participants in therapeutic situations that require cooperative involvement. It is expanding the inclusivity of therapy by accommodating both children and adults in online formats. It is strengthening client motivation by engaging them in visually and auditorily interactive activities. It is providing a more comfortable treatment environment that reduces anxiety associated with in-person sessions.

Existing remote therapy tools are limiting therapeutic depth by relying only on basic audio and video interaction without experiential engagement. They are consistently reducing the therapist's ability to introduce structured emotional exercises during online sessions. They are creating difficulties for clients who struggle to stay engaged in simple conversation formats. They are producing ineffective outcomes for individuals who require multisensory therapeutic involvement. They are preventing counselors from maintaining therapeutic momentum as clients lose focus easily online. They are lowering the impact of therapy by restricting the session to passive communication.

Existing remote therapy systems are fragmenting therapy workflows by forcing therapists to switch between multiple unrelated digital tools. They are slowing down therapy progress because materials and instructions exist across separate platforms. They are generating confusion for clients who need clear and unified direction to emotionally participate. They are complicating session management by requiring manual organization of activities during therapy. They are adding technical friction that distracts both clients and counselors from emotional work. They are decreasing efficiency by dividing attention between therapy and technology troubleshooting.

Existing digital therapy environments are excluding clients who benefit from creative and interactive therapeutic methods. They are restricting remote therapy to verbal conversation, reducing inclusivity for children and neurodivergent clients. They are offering limited support for immersive emotional expression during online sessions. They are reducing the diversity of therapy styles available to counselors in remote environments. They are forcing therapists to abandon experiential approaches when interacting through digital platforms. They are diminishing the therapist's control over therapeutic engagement and immersion during online counseling.

US20150324532 is describing a virtual mental health platform that focuses on matching a patient with suitable therapists, delivering initial assessments, and managing continuous care plans through remote communication. This prior art is centering its innovation on automated matching algorithms, categorization of users, and progress tracking, rather than on rich experiential therapeutic work inside the session itself. As a result, it is primarily optimizing intake and administrative aspects of therapy and is not addressing the need for structured, interactive therapeutic activities that support real time emotional processing between counselor and client during live sessions.

US20190206518 is disclosing a method of providing online mental health care that relies on therapist and user applications running on separate client devices connected through a server. The focus is on enabling communication and data exchange across geographical distance by means of conventional telemedicine style interactions. This prior art is largely framing therapy as a sequence of audio or video consultations and database operations, so it is not resolving the challenge of delivering diverse experiential modalities in a unified, session centered environment. As a result, it is leaving therapists to improvise experiential work with external tools that are not coordinated with the therapeutic flow of the online session.

US20220068158 is directed to systems and methods for providing mental distress therapy through interaction with an interactive space. The disclosure is oriented toward a specific style of interactive environment for distress reduction and concentrates on the structure of that single experiential space. It is not providing a general framework that supports multiple different experiential techniques within one coherent platform that a human counselor is orchestrating. Consequently, it is not addressing the practical requirement for therapists to shift fluidly between different experiential approaches, populations, and session goals while maintaining continuity within a single online therapeutic setting.

US20220230766 is directed to a system for providing a remote therapy training program, particularly for eye movement desensitization and reprocessing and related bilateral stimulation systems. The prior art is emphasizing how trainers and therapists interact with a training environment in order to learn to use a specific therapy system effectively. It is therefore primarily focused on professional training logistics instead of ongoing client treatment workflows. This means it is not resolving the broader problem of delivering actual multifaceted therapy sessions to clients in a live telehealth context where multiple experiential approaches require coordination in real time.

CN117412702A is describing a system and method for psychological treatment based on artificial intelligence psychotherapy. The disclosure is centering on obtaining patient messages, using classifiers, and generating automated responses based on artificial intelligence techniques. This approach is largely replacing or supplementing human therapists with artificial intelligence driven dialogue and classification, and it addresses the problem of automated scalable support. However, it is not solving the need for a human counselor to guide rich, collaborative experiential work with clients through an online environment, and it is not structuring multi person interactive activities that reflect traditional in person therapy modalities.

CN120299634B is directed to a guiding auxiliary platform based on mental health education that supports mental health teaching, assessment, and guidance. The invention is focused on the context of education and mental health promotion rather than on full depth clinical therapy sessions. Its functions relate to educational testing, guidance, and support for mental health education settings, not to intensive trauma processing or complex experiential counseling workflows. As a result, it is not addressing the need for a comprehensive online environment where licensed therapists are continuously conducting structured experiential therapy with individuals, families, or children in real time.

The present invention is addressing these limitations by providing a unified online therapeutic framework that is organizing remote counseling sessions around structured experiential interactions rather than around basic communication or administrative matching functions alone. The invention is supporting human therapists in conducting rich, emotionally meaningful work through an online environment that is purpose built for counseling, so that the remote session is evolving as an active therapeutic experience rather than a simple video conversation. By treating the session as a coordinated sequence of guided experiential directives, the invention is enabling deeper engagement, clearer therapeutic boundaries, and more consistent emotional processing, while remaining compatible with existing telehealth infrastructure and privacy expectations.

The invention is also addressing fragmentation across existing solutions by placing diverse experiential approaches under one coherent digital umbrella that is designed for real time collaboration between counselors, clients, and where relevant additional participants. Instead of requiring therapists to jump between unrelated software tools, the invention is enabling them to remain within a single environment that is supporting planning, delivery, and closure of complex therapeutic encounters. This integrated approach is reducing cognitive load on both counselor and client, is maintaining therapeutic momentum, and is creating a stable container in which experiential work is taking place safely and predictably during online sessions, regardless of the specific therapeutic modality being applied.

### SUMMARY OF THE INVENTION

Embodiments of the present invention relate to an online multifaceted therapy delivery system. The system comprising a user device configured to receive experiential therapy content, display interactive activities, capture user responses, and transmit audiovisual information during a live online therapy session, wherein the user device comprises a display unit configured to visually render experiential therapy content, visual stimulation, interactive activities, and counselor-driven experiential instructions during the live therapy session, a microphone configured to capture verbal responses of the user and transmit the verbal responses to a counselor through the system, an image capturing unit configured to record visual information of the user and transmit the visual information to the counselor through the system, a speaker unit configured to output auditory stimulation during experiential therapy activities, a touch interface configured to detect drawing, object manipulation, and activity interaction on the displayed experiential therapy content, a keyboard configured to receive typed inputs from the user for expressive therapy activities, an eye tracking sensor configured to detect eye movements of the user and transmit the eye movement data to the counselor through the system. The system also comprising an application server connected to the user device, configured to execute therapy session orchestration instructions, coordinate audiovisual streaming between the user and the counselor, allocate experiential therapy modules during the live therapy session, and maintain uninterrupted synchronization of therapy activities between the user and the counselor. The system also comprising a database connected to the application server, configured to store experiential therapy modules, experiential therapy activity scripts, session access information, stimulus control rules, synchronization rules, and session log information. The system also comprising a communication network connected to the application server and to the user device, configured to transfer the audiovisual information, the eye movement data, the touch interaction information, the typed inputs, the therapy module instructions, the synchronization information, and the session commands between the user device and the application server in real time. The system also comprising a processing unit connected to the application server through the communication network, configured to interpret and execute experiential therapy operations during the live therapy session, wherein the processing unit comprises a session management module connected to the application server and configured to verify session access, activate the therapy sessions, and terminate the therapy sessions when instructed by the counselor, a synchronization control module connected to the user device and configured to generate corresponding experiential events for every counselor command during the live therapy session, a media stimulus delivery module connected to the display unit and to the speaker unit and configured to deliver visual stimulation, auditory stimulation, and bilateral experiential therapy activity content to the user, a data logging module connected to the database and configured to record user interaction activity, audiovisual activity, and sensor generated activity during the live therapy session. The system also comprising a user interface connected to the processing unit and user device and configured to display counselor-driven experiential instructions to the user and to transmit user interaction information back to the processing unit in real time.

In accordance with an embodiment of the present invention, the system further comprises a counselor interface connected to the synchronization control module through the application interface and configured to allow the counselor to issue real time experiential instructions that generate experiential events on the user device.

In accordance with an embodiment of the present invention, the processing unit further comprises a session link generation module connected to the session management module and configured to generate a unique session access link that associates a selected experiential therapy module with a therapy session.

In accordance with an embodiment of the present invention, the processing unit further comprises a smart computational artificial intelligence module configured to analyze sensor-generated activity and experiential interaction patterns and to generate a summarized experiential profile for the counselor to review.

In accordance with an embodiment of the present invention, the database further stores a library of experiential therapy modules for different counseling modalities, wherein each experiential therapy module being retrievable and executable during the live therapy session.

In accordance with an embodiment of the present invention, the communication network is further configured to transfer multi-participant audiovisual information, allowing multiple users to join the therapy session simultaneously under the control of the counselor.

In accordance with an embodiment of the present invention, the synchronization control module is further configured to switch between multiple experiential therapy modules during one uninterrupted therapy session without terminating the session.

In accordance with an embodiment of the present invention, the media stimulus delivery module is further configured to deliver bilateral visual stimulation and bilateral auditory stimulation in response to commands received from the counselor interface.

In accordance with an embodiment of the present invention, the data logging module is further configured to store time-stamped interaction events including drawing activity, object placement activity, movement activity, and keyboard expressive activity inside the database.

Another embodiment of the present invention relates to a process for online multifaceted therapy delivery and the method comprising receiving therapist authentication credentials through a user interface and transferring the therapist authentication credentials through a communication network to a processing unit of an application server for verification through a session management module of the processing unit. The method also comprising retrieving a library of experiential therapy modules from a database connected to the application server and displaying the library of experiential therapy modules on a counselor interface connected to the synchronization control module of the processing unit to enable therapist selection of an experiential therapy module. The method also comprising generating a unique session access link through a session link generation module of the processing unit and transmitting the unique session access link to a user device through the communication network for activation of the experiential therapy module during a live therapy session. The method also comprising establishing audiovisual communication by receiving microphone information, visual information from an image capturing unit, keyboard inputs, touch interaction information, and eye movement information from the user device and transmitting the received information through the communication network to the processing unit of the application server for execution of the experiential therapy operations. The method also comprising rendering experiential therapy content by delivering visual stimulation on a display unit of the user device and delivering auditory stimulation through a speaker unit of the user device through a media stimulus delivery module of the processing unit in response to live experiential commands originating from the counselor interface. The method also comprising generating synchronized experiential events by mapping each experiential command from the counselor interface to a corresponding experiential activity on the user device through the synchronization control module of the processing unit during the therapy session. The method also comprising executing a continuous experiential therapy loop by receiving real time user interaction information from the touch interface, the keyboard, the microphone, the image capturing unit, and the eye tracking sensor and transmitting the real time user interaction information to the processing unit for live modification of experiential stimuli through the synchronization control module. The method also comprising maintaining uninterrupted experiential interaction through sequential switching between multiple experiential therapy modules during a single therapy session without exiting the therapy session and without terminating audiovisual communication. The method also comprising processing user interaction information, audiovisual information, and sensor generated information through a smart computational artificial intelligence module of the processing unit for generation of experiential outcome information and transmitting the experiential outcome information to the counselor interface for review. The method also comprising recording therapy progression by storing time stamped audiovisual information, therapist command information, experiential interaction information, sensor generated information, and experiential outcome information inside the database through a data logging module of the processing unit. The method also comprising terminating the therapy session by receiving a termination command from the counselor interface and executing session closure through the session management module for final storage of therapy session information in the database.

In accordance with an embodiment of the present invention, the process further comprising displaying counselor driven experiential instructions on the user interface of the user device and receiving user expressive responses through the user interface for transmission to the processing unit for real time therapy progression control.

In accordance with an embodiment of the present invention, the process further comprising receiving multi-participant audiovisual information from two or more user devices through the communication network and synchronizing the experiential activities across multiple user devices under the control of the counselor interface during the live therapy session.

In accordance with an embodiment of the present invention, the process further comprising executing an experiential therapy activity that involves interactive drawing across multiple users by receiving sequential line-drawing interaction information from multiple user devices and merging the line-drawing interaction information into a single shared experiential drawing output through the synchronization control module.

In accordance with an embodiment of the present invention, the process further comprising delivering bilateral stimulation during the experiential therapy content by transmitting alternating visual stimulation and alternating auditory stimulation to the display unit and the speaker unit in direct response to bilateral stimulation instructions issued through the counselor interface.

In accordance with an embodiment of the present invention, the process further comprising receiving selection of expressive activity assets from a graphical asset library stored in the database and rendering the expressive activity assets in an object manipulation workspace on the display unit of the user device for experiential therapy involvement.

In accordance with an embodiment of the present invention, the process further comprising generating an experiential mapping display that includes labeled imagery provided by the client and transmitting the labeled imagery mapping display to the counselor interface for real time review during the live therapy session.

In accordance with an embodiment of the present invention, the process further comprising executing a mindfulness experiential activity by receiving sound identification responses from the user through the microphone and the keyboard and transmitting the sound identification responses to the processing unit for validation through the smart computational artificial intelligence module.

In accordance with an embodiment of the present invention, the process further comprising receiving continuous bilateral drawing activity information from the touch interface and from keyboard directional keys of the user device and converting the bilateral drawing activity information into an abstract experiential drawing display through the media stimulus delivery module.

In accordance with an embodiment of the present invention, the process further comprising receiving modification instructions from the counselor interface to alter the experiential therapy module currently in use and switching to another experiential therapy module while preserving the experiential interaction state already produced during the session.

In accordance with an embodiment of the present invention, the process further comprising generating a final experiential record by compiling the time stamped audiovisual information, therapist command information, experiential interaction information, sensor generated information, and experiential outcome information into an exportable file through the data logging module during completion of session closure.

### BRIEF DESCRIPTION OF THE DRAWINGS

So that the manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

The invention herein will be better understood from the following description with reference to the drawings, in which:

FIG. 1 illustrates a block diagram for an online multifaceted therapy delivery system and process thereof, in accordance with an embodiment of the present invention;

FIG. 2 illustrates a flowchart for a process for online multifaceted therapy delivery, in accordance with an embodiment of the present invention;

FIG. 3A illustrates an introductory interface for initiating online multifaceted therapy delivery, in accordance with an embodiment of the present invention;

FIG. 3B illustrates an authentication interface for therapist or client sign-in, in accordance with an embodiment of the present invention;

FIG. 3C illustrates an account creation interface for system registration, in accordance with an embodiment of the present invention;

FIG. 4A illustrates an experiential therapy module selection interface, in accordance with an embodiment of the present invention;

FIG. 4B illustrates a counselor monitoring and directive control interface, in accordance with an embodiment of the present invention;

FIG. 5A illustrates a connection management interface for linking counselor and client devices, in accordance with an embodiment of the present invention;

FIG. 5B illustrates a session timeout alert and reconnection interface, in accordance with an embodiment of the present invention;

FIG. 6A illustrates an audio initiation interface for experiential therapy activation, in accordance with an embodiment of the present invention;

FIG. 6B illustrates an audio control interface managed by the counselor during an experiential session, in accordance with an embodiment of the present invention;

FIG. 6C illustrates an experiential auditory-visual control interface for EMDR therapy, in accordance with an embodiment of the present invention;

FIG. 7A illustrates an online sand tray experiential workspace rendered within a therapy session, in accordance with an embodiment of the present invention;

FIG. 7B illustrates an instructional prompt shown to guide user interaction during an expressive experiential task, in accordance with an embodiment of the present invention; and

FIG. 7C illustrates an experiential drawing interface generated during a collaborative or bilateral drawing directive, in accordance with an embodiment of the present invention.

It should be noted that the accompanying figure is intended to present illustrations of exemplary embodiments of the present disclosure. This figure is not intended to limit the scope of the present disclosure. It should also be noted that the accompanying figure is not necessarily drawn to scale.

### DETAILED DESCRIPTION OF THE INVENTION

In the following detailed description, numerous specific details are set forth in order to provide a thorough understanding of the embodiment of the invention as illustrative or exemplary embodiments of the invention, specific embodiments in which the invention may be practiced are described in sufficient detail to enable those skilled in the art to practice the disclosed embodiments. However, it will be obvious to a person skilled in the art that the embodiments of the invention may be practiced with or without these specific details. In other instances, well-known methods, procedures and components have not been described in detail so as not to unnecessarily obscure aspects of the embodiments of the invention.

The following detailed description is, therefore, not to be taken in a limiting sense, and the scope of the present invention is defined by the appended claims and equivalents thereof. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. References within the specification to “one embodiment,” “an embodiment,” “embodiments,” or “one or more embodiments” are intended to indicate that a particular feature, structure, or characteristic described in connection with the embodiment is included in at least one embodiment of the present invention.

Although the terms first, second, etc. may be used herein to describe various elements, these elements should not be limited by these terms. These terms are generally only used to distinguish one element from another and do not denote any order, ranking, quantity, or importance, but rather are used to distinguish one element from another. Further, the terms “a” and “a” herein do not denote a limitation of quantity, but rather denote the presence of at least one of the referenced items.

The conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The following brief definition of terms shall apply throughout the present invention

The terms “determining”, “measuring”, “evaluating”, “assessing,” “assaying,” and “analyzing” can be used interchangeably herein to refer to any form of measurement, and include determining if an element is present or not. (e.g., detection). These terms can include both quantitative and/or qualitative determinations. Assessing may be relative or absolute.

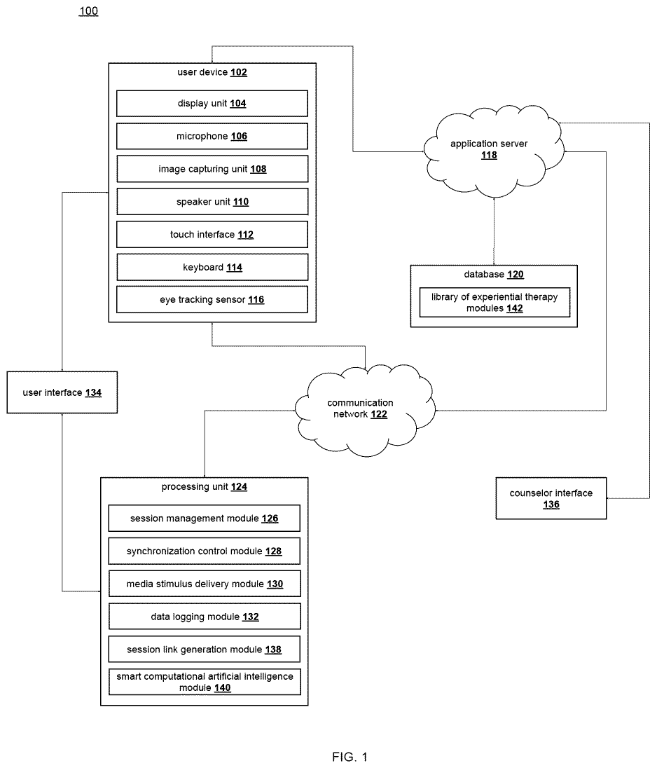

FIG. 1 illustrates a block diagram for an online multifaceted therapy delivery system and process thereof, in accordance with an embodiment of the present invention.

The user device 102 operates as an interactive therapeutic terminal that continuously receives experiential therapy content, renders multisensory outputs, and transmits user-generated responses during an online therapy session. The user device 102 maintains seamless communication with the application server 118 through the communication network 122, allowing all experiential therapy modules 142 to execute without interruption. The architecture of the user device 102 includes multiple components that collectively facilitate expressive, sensory, cognitive, and motor-based engagement from the user during real time experiential activities.

The display unit 104 functions as the visual rendering surface of the user device 102 and presents every experiential therapy interaction generated during the live therapeutic session. The display unit 104 visually conveys experiential therapy content, visual stimulation sequences, drawing workspaces, sand tray surfaces, expressive mapping layouts, comic cell environments, bilateral stimulus cues, emotional cards, and all counselor-driven experiential instructions transmitted through the synchronization control module 128. The display unit 104 maintains consistent alignment between therapist instructions and user interactions by presenting updated activity states during every experiential transition.

The microphone 106 captures verbal responses produced by the user during the therapy session and transfers the captured audio information to the application server 118 for real time delivery to the counselor interface 136. The microphone 106 also supports experiential tasks that involve identifying sounds, responding verbally to sensory cues, or describing emotional narratives that form part of the therapy progression.

The image capturing unit 108 records visual information of the user and continuously transmits this visual information to the processing unit 124 through the communication network 122. The image capturing unit 108 tracks facial expressions, body posture variations, and moment-by-moment reactions that may be relevant during expressive activities, narrative tasks, or trauma-processing experiential modules.

The speaker unit 110 outputs auditory stimulation associated with experiential therapy activities. The speaker unit 110 renders bilateral tones, alternating auditory cues, musical patterns, mindfulness sounds, and counselor-issued voice instructions. The audio output remains synchronized with visual cues delivered by the display unit 104 through the coordination of the media stimulus delivery module 130.

The touch interface 112 detects real time tactile interactions made by the user on the experiential therapy content displayed on the display unit 104. The touch interface 112 captures drawing strokes, object placements, emoji arrangements, symbolic manipulations, bilateral drawing motions, sand tray reshaping actions, and expressive design elements that form essential components of several experiential therapy modules 142.

The keyboard 114 receives typed user inputs during expressive therapy tasks that involve labeling internal thoughts, entering emotional descriptors, composing values, creating story components, or writing narrative text. The keyboard 114 also supports directional commands during bilateral drawing exercises and interactive storytelling activities.

The eye tracking sensor 116 measures eye movement patterns performed by the user, including saccadic motions, fixation behavior, bilateral tracking movements, and changes in visual focus. The eye tracking sensor 116 sends the recorded eye movement data to the processing unit 124, where it assists in aligning bilateral visual stimulation, assessing engagement levels, and supporting experiential modules that require gaze-based tracking.

In another embodiment of the present disclosure, the user device 102 integrates haptic feedback systems that generate vibration-based sensory cues during experiential transitions to support grounding or sensory regulation tasks.

In another embodiment of the present disclosure, the display unit 104 presents stereoscopic or depth-enhanced graphics for immersive experiential therapy activities such as three-dimensional sand tray arrangements or layered expressive mapping.

In another embodiment of the present disclosure, the microphone 106 records multi-channel audio input to support group therapy sessions where multiple participants communicate through separate audio streams routed by the application server 118.

In another embodiment of the present disclosure, the image capturing unit 108 incorporates depth sensors that track upper-body movement for advanced expressive tasks where body posture and gesture formation contribute to therapeutic expression.

In another embodiment of the present disclosure, the touch interface 112 includes pressure-sensing capabilities for granular sand manipulation, symbolic weighting tasks, and delicate expressive motions that require tracking of applied force.

In another embodiment of the present disclosure, the keyboard 114 includes expandable on-screen emotional vocabulary panels that support symbolic labeling during expressive therapy.

In another embodiment of the present disclosure, the eye tracking sensor 116 performs simultaneous pupil dilation measurement to provide additional behavioral signals relevant during trauma stabilization or emotional regulation exercises.

The system 100 may include a user device 102 configured to receive experiential therapy content, display interactive activities, capture user responses, and transmit audiovisual information during a live online therapy session, wherein the user device 102 comprises a display unit 104 configured to visually render experiential therapy content, visual stimulation, interactive activities, and counselor-driven experiential instructions during the live therapy session, a microphone 106 configured to capture verbal responses of the user and transmit the verbal responses to a counselor through the system 100, an image capturing unit 108 configured to record visual information of the user and transmit the visual information to the counselor through the system 100, a speaker unit 110 configured to output auditory stimulation during experiential therapy activities, a touch interface 112 configured to detect drawing, object manipulation, and activity interaction on the displayed experiential therapy content, a keyboard 114 configured to receive typed inputs from the user for expressive therapy activities, an eye tracking sensor 116 configured to detect eye movements of the user and transmit the eye movement data to the counselor through the system 100.

The application server 118 functions as the central computational infrastructure that governs orchestration, coordination, and real time execution of all experiential therapy operations within the system 100. The application server 118 remains continuously connected to the user device 102 through the communication network 122 and receives every form of input generated during the live session, including verbal information from the microphone 106, visual information from the image capturing unit 108, typed inputs from the keyboard 114, interaction data from the touch interface 112, and gaze tracking information from the eye tracking sensor 116. The application server 118 processes this information and synchronizes it with therapist-generated instructions received through the counselor interface 136, ensuring that the experiential therapy module currently active aligns with the therapist's moment-to-moment directives. The application server 118 executes therapy session orchestration instructions by managing initiation, progression, real time updating, and termination of every experiential therapy module 142 selected by the therapist.

The application server 118 communicates directly with the database 120 to retrieve experiential therapy modules, therapy activity scripts, stimulus control rules, synchronization rules, and associated session-access data. During an active therapy session, the application server 118 transmits all visual stimulation and auditory stimulation commands to the media stimulus delivery module 130 for rendering on the user device 102. The application server 118 also communicates with the synchronization control module 128 to generate immediate experiential events in direct response to therapist commands. Through its processing unit 124, the application server 118 maintains uninterrupted synchronization between the therapist's actions and the experiential responses displayed on the user device 102. The application server 118 further supports multi-participant therapy operations by routing audiovisual streams and experiential interactions across several user device 102 units during collaborative or family-based therapeutic scenarios.

In another embodiment of the present disclosure, the application server 118 utilizes load-balancing techniques to dynamically distribute experiential processing tasks across multiple parallel server instances to ensure stable performance during high-demand interactive sessions.

In another embodiment of the present disclosure, the application server 118 incorporates an adaptive session mapping engine that modifies experiential module behavior based on cumulative sensor patterns processed through the smart computational artificial intelligence module 140.

In another embodiment of the present disclosure, the application server 118 maintains encrypted parallel data channels to support confidential, multi-channel experiential feedback for high-sensitivity therapeutic interactions.

The system 100 may include an application server 118 connected to the user device 102, configured to execute therapy session orchestration instructions, coordinate audiovisual streaming between the user and the counselor, allocate experiential therapy modules during the live therapy session, and maintain uninterrupted synchronization of therapy activities between the user and the counselor.

The database 120 operates as the centralized storage structure that maintains all experiential, functional, and session-oriented data required for the operation of the system 100. The database 120 remains connected to the application server 118 and continuously exchanges information needed for retrieval, execution, modification, and archival of experiential therapy content. The database 120 stores experiential therapy modules 142, therapy activity scripts, stimulus control rules, synchronization rules, session access information, and session log information in formats optimized for rapid retrieval during live interactive use. The database 120 supports bidirectional data movement between the application server 118 and the processing unit 124 so that the system 100 maintains uninterrupted presentation of experiential content, even while the user device 102 generates significant quantities of real time interaction data.

The database 120 organizes every experiential therapy module 142 in a structured manner that enables session management module 126, synchronization control module 128, and media stimulus delivery module 130 to access the correct content, stimuli, and logic required at any given stage of the therapy session. Each experiential therapy module 142 stored in the database 120 includes layered activity logic, directive structures, dynamic sequencing rules, and content resources that define its therapeutic behavior. Experiential therapy modules 142 include drawing-based experiential activities, sand tray arrangements, expressive mapping scenes, bilateral stimulation routines, musical or auditory mindfulness tasks, emotional card activities, collaborative drawing exercises, symbolic transformation tasks, multi-participant activities, and structured narrative-building interactions. These experiential therapy modules 142 are stored in a modular format so they are individually retrievable and executable during a live session and are capable of being combined or transitioned during a single session under direction of the counselor interface 136.

The database 120 also stores session access information that associates each experiential therapy module 142 with a generated session access link created by the session link generation module 138. During the live session, the database 120 records time stamped audiovisual information, therapist command information, experiential interaction information, sensor generated information, and experiential outcome information through the data logging module 132. The archived information stored within the database 120 is used for session review, continuity across future sessions, and preservation of experiential history generated during therapeutic work. The database 120 therefore functions as the persistent memory foundation of the system 100, supporting both real time behavior and long-term data preservation.

In another embodiment of the present disclosure, the database 120 employs parallel indexing and multi-partitioned storage so that experiential therapy modules 142 load sequentially without latency during transitions inside a single session.

In another embodiment of the present disclosure, the database 120 maintains encrypted experiential therapy records that can be selectively revealed to authorized therapists while preserving confidential portions of user-generated expressive content.

In another embodiment of the present disclosure, experiential therapy modules 142 stored in the database 120 include adaptive branching logic that alters module progression based on real time sensor data processed through the smart computational artificial intelligence module 140.

The system 100 may include a database 120 connected to the application server 118, configured to store experiential therapy modules, experiential therapy activity scripts, session access information, stimulus control rules, synchronization rules, and session log information.

The database 120 further stores a library of experiential therapy modules 142 for different counseling modalities, wherein each experiential therapy module being retrievable and executable during the live therapy session.

The communication network 122 operates as the continuous data transmission layer that connects the user device 102 to the application server 118 and supports uninterrupted flow of audiovisual information, interaction information, and experiential commands throughout the therapy session. The communication network 122 transfers microphone 106 audio data, image capturing unit 108 visual data, keyboard 114 typed inputs, touch interface 112 interaction data, and eye tracking sensor 116 gaze information from the user device 102 to the processing unit 124 in real time. The communication network 122 also carries the stimulus control instructions, synchronization information, experiential module updates, counselor-driven experiential commands, and module transition signals from the application server 118 back to the user device 102. The communication network 122 maintains stable, low-latency bidirectional communication so that every experiential event generated through the synchronization control module 128 is accurately mirrored on the user device 102. The communication network 122 additionally supports multi-participant connectivity by routing audiovisual information and experiential activity information across multiple user device 102 units during collaborative therapy sessions controlled through the counselor interface 136. The communication network 122 therefore functions as the dynamic transport medium that enables the high-speed interactive behavior essential to the experiential therapy modules 142.

In another embodiment of the present disclosure, the communication network 122 employs prioritized routing for experiential commands so that synchronization instructions and bilateral stimulation signals receive transmission precedence over non-critical data.

In another embodiment of the present disclosure, the communication network 122 integrates adaptive bandwidth allocation that increases throughput to the media stimulus delivery module 130 during high-intensity visual or auditory stimulation sequences.

The system 100 may include a communication network 122 connected to the application server 118 and to the user device 102, configured to transfer the audiovisual information, the eye movement data, the touch interaction information, the typed inputs, the therapy module instructions, the synchronization information, and the session commands between the user device 102 and the application server 118 in real time.

The communication network 122 is further configured to transfer multi-participant audiovisual information, allowing multiple users to join the therapy session simultaneously under the control of the counselor.

The processing unit 124 functions as the integrated computational framework that executes all experiential therapy operations within the system 100. The processing unit 124 remains connected to the application server 118 through the communication network 122 and continuously receives input data generated by the user device 102, including microphone information from the microphone 106, visual information from the image capturing unit 108, typed inputs from the keyboard 114, interaction data from the touch interface 112, and gaze-based information from the eye tracking sensor 116. The processing unit 124 interprets this continuous influx of interaction data and synchronizes it with the therapist-generated experiential commands originating from the counselor interface 136. The processing unit 124 supervises real time execution of experiential therapy modules 142, coordinates timing, updates interactive states, and manages sequencing of activities during the live therapy session.

The session management module 126 is integrated within the processing unit 124 and governs the structural lifecycle of each therapy session. The session management module 126 verifies session access credentials transferred from the user interface 134, activates the live therapy session, links the session to a selected experiential therapy module 142, and manages the termination sequence at the conclusion of the therapy encounter. The session management module 126 ensures that every experiential event, transition, and command processed during the session is anchored to a valid session state, allowing uninterrupted experiential interaction from initiation until closure.

The synchronization control module 128 operates within the processing unit 124 as the component responsible for transforming therapist commands into synchronized experiential events on the user device 102. The synchronization control module 128 receives instructions from the counselor interface 136 and immediately maps each instruction to the corresponding experiential output that appears on the display unit 104 or the speaker unit 110. The synchronization control module 128 updates interactive states in response to real time user movements, drawing activity, eye-tracking patterns, expressive typing, and symbolic manipulations captured through the user device 102. The synchronization control module 128 is further configured to switch between multiple experiential therapy modules 142 during a single uninterrupted session and to maintain continuity between diverse experiential states.

The media stimulus delivery module 130, housed within the processing unit 124, generates all visual and auditory stimuli required for experiential therapy. The media stimulus delivery module 130 transmits visual patterns to the display unit 104, auditory sequences to the speaker unit 110, and bilateral experiential signals that alternate rhythmically for psychological processing tasks. The media stimulus delivery module 130 executes bilateral stimulation routines, guided imagery displays, interactive drawing presentations, symbolic visualization environments, musical or mindfulness-based sequences, and coordinated color or movement cues that support engagement within the experiential therapy modules 142.

The data logging module 132 records every significant experiential event that occurs during the live therapy session. The data logging module 132 stores time stamped audiovisual information, therapist command information, interaction patterns recorded through the touch interface 112, movement-related information received from bilateral drawing activities, typed expressive content entered through the keyboard 114, gaze movement captured by the eye tracking sensor 116, and generated experiential outcomes processed through the smart computational artificial intelligence module 140. The data logging module 132 ensures that every activity performed during the session is securely archived within the database 120 for future review and therapeutic continuity.

The session link generation module 138 generates unique session access links that connect specific user device 102 units to selected experiential therapy modules 142. The session link generation module 138 associates each link with session metadata stored in the database 120, allowing synchronized entry into the experiential environment and enabling instant module activation once the session begins.

The smart computational artificial intelligence module 140 extends the capabilities of the processing unit 124 by analyzing user-generated interaction data, sensor patterns, expressive movements, gaze behavior, and narrative inputs. The smart computational artificial intelligence module 140 interprets emergent experiential patterns and generates experiential outcome information that is transmitted to the counselor interface 136 for real time review. The smart computational artificial intelligence module 140 supports adaptive experiential progression, symbolic categorization tasks, emotional identification routines, and complex multi-layered experiential transformations that enhance interactive therapeutic depth within the experiential therapy modules 142.

In another embodiment of the present disclosure, the processing unit 124 employs distributed computation across multiple processors to maintain stable experiential responsiveness during sessions containing high-intensity visual or auditory sequences.

In another embodiment of the present disclosure, the session management module 126 maintains multi-session continuity so that experiential states, symbolic structures, or story components created during a previous session reappear automatically during future therapy interactions.

In another embodiment of the present disclosure, the synchronization control module 128 dynamically adjusts timing, visual pacing, and stimulus direction in response to real time behavioral patterns detected by the smart computational artificial intelligence module 140.

In another embodiment of the present disclosure, the media stimulus delivery module 130 generates synchronized multi-modal patterns that combine visual color transitions, bilateral tone pulses, and environmental ambience for experiential grounding tasks.

In another embodiment of the present disclosure, the data logging module 132 creates encrypted multi-layered logs that can be partitioned into therapist-accessible and client-accessible sections for controlled information sharing.

In another embodiment of the present disclosure, the session link generation module 138 supports temporary guest participant access links for collaborative therapeutic modules requiring parental or guardian involvement.

In another embodiment of the present disclosure, the smart computational artificial intelligence module 140 generates predictive experiential trajectories based on cumulative user behavior across multiple therapy sessions.

The system 100 may include a processing unit 124 connected to the application server 118 through the communication network 122, configured to interpret and execute experiential therapy operations during the live therapy session, wherein the processing unit 124 comprises a session management module 126 connected to the application server 118 and configured to verify session access, activate the therapy sessions, and terminate the therapy sessions when instructed by the counselor, a synchronization control module 128 connected to the user device 102 and configured to generate corresponding experiential events for every counselor command during the live therapy session, a media stimulus delivery module 130 connected to the display unit 104 and to the speaker unit 110 and configured to deliver visual stimulation, auditory stimulation, and bilateral experiential therapy activity content to the user, a data logging module 132 connected to the database 120 and configured to record user interaction activity, audiovisual activity, and sensor generated activity during the live therapy session.

The processing unit 124 further comprises a session link generation module 138 connected to the session management module 126 and configured to generate a unique session access link that associates a selected experiential therapy module with a therapy session.

The processing unit 124 further comprises a smart computational artificial intelligence module 140 configured to analyze sensor-generated activity and experiential interaction patterns and to generate a summarized experiential profile for the counselor to review.

The synchronization control module 128 is further configured to switch between multiple experiential therapy modules during one uninterrupted therapy session without terminating the session.

The media stimulus delivery module 130 is further configured to deliver bilateral visual stimulation and bilateral auditory stimulation in response to commands received from the counselor interface 136.

The data logging module 132 is further configured to store time-stamped interaction events including drawing activity, object placement activity, movement activity, and keyboard 114 expressive activity inside the database 120.

The user interface 134 serves as the primary interaction environment displayed on the user device 102 through which the user engages with experiential therapy content during the live therapy session. The user interface 134 continuously presents counselor-driven experiential instructions, visual activity cues, symbolic prompts, drawing spaces, object manipulation surfaces, emotional expression elements, bilateral stimulation displays, and narrative construction areas in accordance with the experiential therapy module 142 selected through the counselor interface 136. The user interface 134 operates as the visual and interactive reflection of the experiential events generated through the synchronization control module 128, ensuring that every counselor-issued command is immediately manifested on the display unit 104 of the user device 102. The user interface 134 receives expressive responses from the user through the microphone 106, the image capturing unit 108, the keyboard 114, the touch interface 112, and the eye tracking sensor 116, and transmits the collected interaction information back to the processing unit 124 for real time modification of experiential stimuli. The user interface 134 further supports transitions between multiple experiential therapy modules 142 by updating interactive elements without interrupting the ongoing therapy session, thereby maintaining continuity throughout the experiential sequence managed by the application server 118.

In another embodiment of the present disclosure, the user interface 134 dynamically adapts its layout based on user engagement patterns identified by the smart computational artificial intelligence module 140.

In another embodiment of the present disclosure, the user interface 134 incorporates color-coded emotional regions that guide the user through expressive identification tasks.

In another embodiment of the present disclosure, the user interface 134 automatically switches between immersive, focused, or minimal display modes depending on the experiential therapy module 142 in use.

The system 100 may include a user interface 134 connected to the processing unit 124 and user device 102 and configured to display counselor-driven experiential instructions to the user and to transmit user interaction information back to the processing unit 124 in real time.

The counselor interface 136 operates as the real time command and supervisory environment through which the therapist directs experiential therapy activities during the live session. The counselor interface 136 remains connected to the synchronization control module 128 and receives continuous updates regarding experiential states, user interactions, and sensor-derived information transmitted from the user device 102. The counselor interface 136 displays the active experiential therapy module 142, provides access to module-specific controls, and allows the therapist to issue experiential commands that are immediately converted into corresponding experiential events on the display unit 104 and the speaker unit 110 through the processing unit 124. The counselor interface 136 enables the therapist to select experiential therapy modules 142, adjust stimulus intensity, alter visual or auditory patterns, trigger bilateral stimulation sequences, initiate expressive mapping tasks, enable collaborative interactions, and transition between experiential states without interrupting audiovisual communication. The counselor interface 136 additionally receives experiential outcome information generated through the smart computational artificial intelligence module 140 and displays the analyzed interaction patterns to support therapeutic interpretation. The counselor interface 136 therefore provides the therapist with direct, moment-to-moment control over all interactive, sensory, and narrative components of the therapy experience.

In another embodiment of the present disclosure, the counselor interface 136 includes predictive experiential guidance panels generated by the smart computational artificial intelligence module 140 to help therapists determine suitable experiential transitions.

In another embodiment of the present disclosure, the counselor interface 136 incorporates collapsible multi-user monitoring windows that allow the therapist to supervise simultaneous activity during group or family therapy modules.

In another embodiment of the present disclosure, the counselor interface 136 supports customizable therapist toolbars that permit rearrangement of experiential controls based on therapeutic style or session preference.

The system 100 further comprises a counselor interface 136 connected to the synchronization control module 128 through the application interface and configured to allow the counselor to issue real time experiential instructions that generate experiential events on the user device 102.

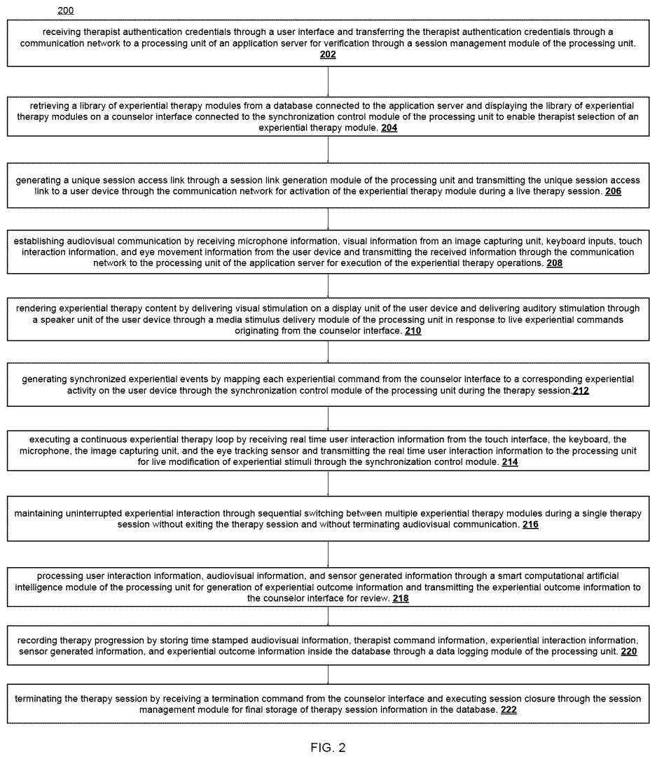

FIG. 2 illustrates a flowchart for a process for online multifaceted therapy delivery, in accordance with an embodiment of the present invention.

At 202, receiving therapist authentication credentials through a user interface 134 and transferring the therapist authentication credentials through a communication network 122 to a processing unit 124 of an application server 118 for verification through a session management module 126 of the processing unit 124 is establishing authenticated entry into the online multifaceted therapy session. The session management module 126 of the processing unit 124 is validating the therapist authentication credentials and maintaining controlled access to ensure that the experiential therapy operations launch securely and remain connected to verified therapist participation.

At 204, retrieving a library of experiential therapy modules 142 from a database 120 connected to the application server 118 and displaying the library of experiential therapy modules 142 on a counselor interface 136 connected to a synchronization control module 128 of the processing unit 124 is providing the therapist with structured access to selectable experiential therapy modules. The synchronization control module 128 of the processing unit 124 is aligning the displayed experiential therapy modules 142 with system readiness so that the therapist is continuously selecting appropriate experiential content for the therapy session.

At 206, generating a unique session access link through a session link generation module 138 of the processing unit 124 and transmitting the unique session access link to a user device 102 through the communication network 122 is establishing the connection framework required for activating the selected experiential therapy module 142. The session link generation module 138 is associating the unique session access link with session metadata stored within the database 120 to ensure that the user device 102 enters the correct experiential environment during the live therapy session.

At 208, establishing audiovisual communication by receiving microphone 106 information, visual information from an image capturing unit 108, keyboard 114 inputs, touch interaction information, and eye movement information from the user device 102 and transmitting the received information through the communication network 122 to the processing unit 124 of the application server 118 is enabling continuous experiential therapy interaction. The processing unit 124 is supervising the integration of all incoming sensor and interaction information to support real time execution of experiential therapy operations across the experiential therapy modules 142.

At 210, rendering experiential therapy content by delivering visual stimulation on a display unit 104 of the user device 102 and delivering auditory stimulation through a speaker unit 110 of the user device 102 through a media stimulus delivery module 130 of the processing unit 124 in response to live experiential commands originating from the counselor interface 136 is creating multisensory experiential engagement. The media stimulus delivery module 130 is aligning visual stimulation and auditory stimulation with therapist-directed experiential progression to support coherent experiential therapy interactions.

At 212, generating synchronized experiential events by mapping each experiential command from the counselor interface 136 to a corresponding experiential activity on the user device 102 through the synchronization control module 128 of the processing unit 124 during the therapy session is forming real time experiential alignment. The synchronization control module 128 is coordinating every experiential transition by translating therapist instructions into immediate visual, auditory, or interactive responses displayed on the display unit 104 and rendered through the speaker unit 110 of the user device 102.

At 214, executing a continuous experiential therapy loop by receiving real time user interaction information from the touch interface 112, the keyboard 114, the microphone 106, the image capturing unit 108, and the eye tracking sensor 116 and transmitting the real time user interaction information to the processing unit 124 for live modification of experiential stimuli through the synchronization control module 128 is supporting uninterrupted experiential adaptability. The synchronization control module 128 is continuously adjusting experiential activity behavior based on user interaction information to maintain therapeutic immersion.

At 216, maintaining uninterrupted experiential interaction through sequential switching between multiple experiential therapy modules 142 during a single therapy session without exiting the therapy session and without terminating audiovisual communication is establishing fluid experiential continuity. The processing unit 124 in coordination with the synchronization control module 128 is ensuring that transitions across experiential therapy modules 142 occur cohesively so that the user device 102 receives uninterrupted visual stimulation, auditory stimulation, and interactive functionality throughout the live therapy session.

At 218, processing user interaction information, audiovisual information, and sensor generated information through a smart computational artificial intelligence module 140 of the processing unit 124 for generation of experiential outcome information and transmitting the experiential outcome information to the counselor interface 136 for review is enabling informed therapeutic evaluation. The smart computational artificial intelligence module 140 is interpreting cumulative experiential patterns and shaping experiential outcome information that supports real time therapeutic decision-making by the therapist.

At 220, recording therapy progression by storing time stamped audiovisual information, therapist command information, experiential interaction information, sensor generated information, and experiential outcome information inside the database 120 through a data logging module 132 of the processing unit 124 is preserving complete experiential session history. The data logging module 132 is structuring the recorded information for future therapeutic review and consistency across subsequent therapy sessions.

At 222, terminating the therapy session by receiving a termination command from the counselor interface 136 and executing session closure through the session management module 126 of the processing unit 124 for final storage of therapy session information in the database 120 is completing the structured conclusion of the online multifaceted therapy session. The session management module 126 is formalizing session closure while ensuring that all experiential information remains securely archived.

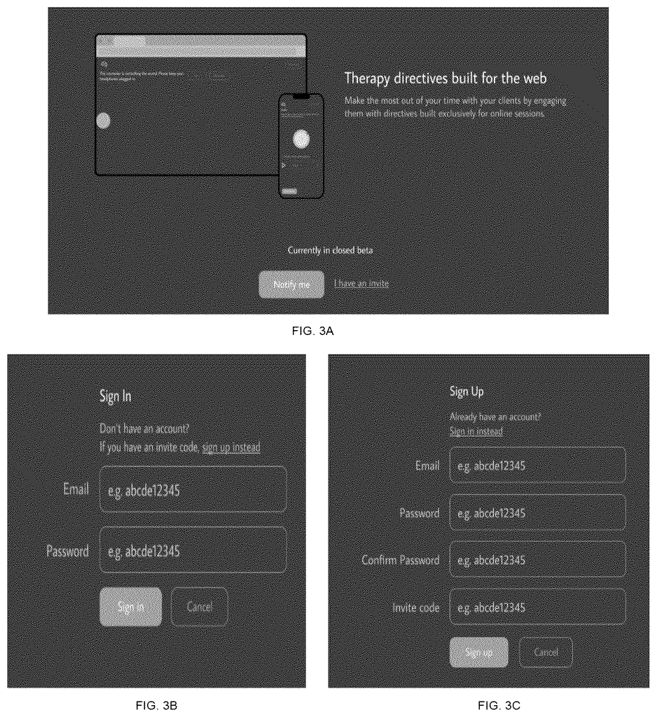

FIG. 3A illustrates an introductory interface for initiating online multifaceted therapy delivery, in accordance with an embodiment of the present invention.

The introductory interface displayed on a user device 102 that is presenting a welcoming environment configured for entry into the online multifaceted therapy delivery system 100. The display unit 104 of the user device 102 is rendering promotional content and session entry prompts that align with the workflow governed through the application server 118 and the processing unit 124. The user interface 134 displayed through the display unit 104 is presenting structured elements that prepare the user for authentication and interaction with experiential therapy modules 142 stored in the database 120. Communication between the user device 102 and the application server 118 through the communication network 122 is continuously preparing the system 100 to transition the user into the live therapy process 200. The counselor interface 136 and related server-side components remain in a standby state as the system 100 is rendering invitation options, access notifications, and activation pathways that harmonize with the session management module 126 and the session link generation module 138. FIG. 3A is therefore presenting the preliminary screen that introduces the user to the controlled, structured environment through which experiential therapy interactions begin forming.

FIG. 3B illustrates an authentication interface for therapist or client sign-in, in accordance with an embodiment of the present invention.

The sign-in environment presented on the display unit 104 of the user device 102, where the user interface 134 is continuously receiving authentication inputs that are processed through the communication network 122 and transferred to the processing unit 124 of the application server 118. The keyboard 114 of the user device 102 is receiving typed authentication data, while the session management module 126 is verifying the authentication credentials to activate a therapy session. The user interface 134 presented through the display unit 104 is coordinating with the synchronization control module 128 to transition from the sign-in phase toward selection and activation of experiential therapy modules 142 once access is confirmed. FIG. 3B is further displaying structured input zones that support secure entry into the system 100, creating a condition where the application server 118, the database 120, and the processing unit 124 operate together to establish a controlled access pathway aligned with the process 200 steps.

FIG. 3C illustrates an account creation interface for system registration, in accordance with an embodiment of the present invention.

The user interface 134 rendered on the display unit 104 of the user device 102 for account creation within the online multifaceted therapy delivery system 100. The user interface 134 is receiving typed information through the keyboard 114 and is transferring the registration data through the communication network 122 to the application server 118, where the processing unit 124 and the session management module 126 are establishing new user access profiles. The counselor interface 136 is being configured for therapist-specific registration when required, while the session link generation module 138 integrates registration information with future session access logic. The database 120 is storing user credentials, session associations, experiential therapy module 142 access permissions, and related metadata required for interaction with the experiential therapy environment. FIG. 3C demonstrates the interface layout through which input fields, verification sequences, and registration elements are being structured to support streamlined entry into the therapy session framework of process 200.

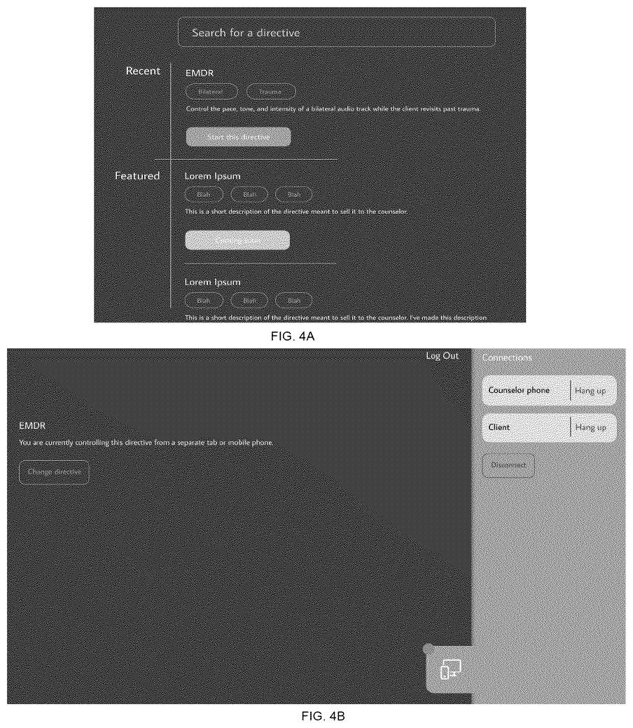

FIG. 4A illustrates an experiential therapy module selection interface, in accordance with an embodiment of the present invention.

The display unit 104 of a user device 102 rendering an interactive environment through the user interface 134, where the user is accessing a structured library of experiential therapy modules 142 stored within the database 120 and retrieved through the application server 118. The display unit 104 is presenting a search bar, recent experiential therapy modules 142, and featured experiential therapy modules 142, each aligned with the synchronization control module 128 of the processing unit 124 to ensure that the user always interacts with session-ready content. The user interface 134 displayed in FIG. 4A is integrating textual descriptions, interactive buttons, and session activation elements so that the user device 102 smoothly transitions into the selected experiential therapy module 142 once the session link generation module 138 of the processing unit 124 prepares the corresponding session access pathway. The structure shown in FIG. 4A is continuously coordinating with the counselor interface 136 and the session management module 126 to maintain real time readiness for immediate entry into the live therapy environment governed by the process 200.

FIG. 4B illustrates a counselor monitoring and directive control interface, in accordance with an embodiment of the present invention.

a counselor-facing environment rendered on the display unit 104 of a user device 102 or a therapist-operated terminal, where the counselor interface 136 is presenting controls used for directing experiential therapy interactions. The display unit 104 is showing session status information, directive selection elements, and connection management controls, which are processed through the communication network 122 and synchronized through the synchronization control module 128 of the processing unit 124. The counselor interface 136 displayed in FIG. 4B remains linked with the application server 118, allowing the therapist to initiate changes to experiential therapy modules 142, adjust sensory outputs managed through the media stimulus delivery module 130, and oversee all audiovisual communication originating from the microphone 106, image capturing unit 108, and speaker unit 110 of the user device 102. FIG. 4B is demonstrating a coordinated interaction space where the session management module 126, the data logging module 132, and the smart computational artificial intelligence module 140 continually support therapist supervision throughout the live therapy session.

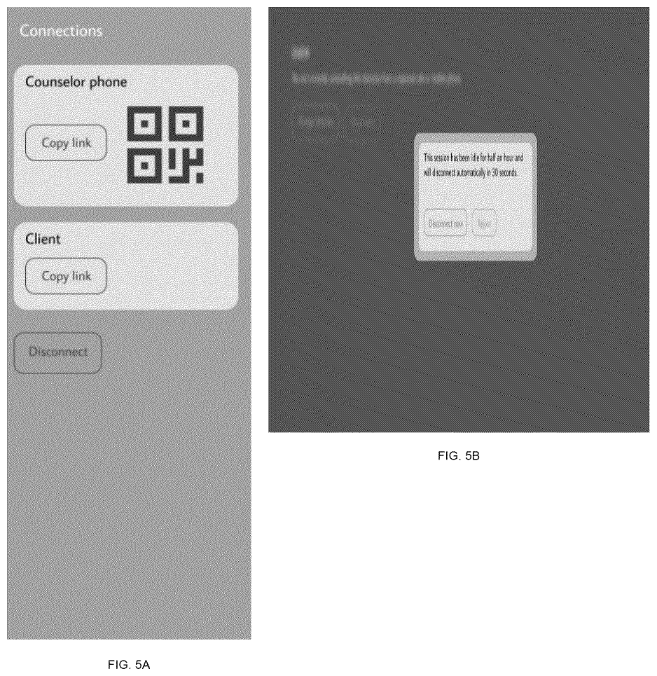

FIG. 5A illustrates a connection management interface for linking counselor and client devices, in accordance with an embodiment of the present invention.

The user interface 134 rendered on the display unit 104 of a user device 102, showing dedicated connection controls for establishing communication pathways between counselor devices and client devices. The user interface 134 is presenting actionable elements that generate session joining links produced through the session link generation module 138 of the processing unit 124 and transmitted through the communication network 122. The database 120 is storing the connection information associated with each generated session link, ensuring that the application server 118 consistently aligns the user device 102 with the correct experiential therapy module 142 when the session begins. The counselor interface 136 and user interface 134 displayed in FIG. 5A operate together with the synchronization control module 128 to maintain continuous audiovisual compatibility with the microphone 106, the image capturing unit 108, and the speaker unit 110 during the live therapy process 200. FIG. 5A further demonstrates an interactive environment where disconnection and reconnection operations are handled through the session management module 126, supporting uninterrupted therapeutic communication.

FIG. 5B illustrates a session timeout alert and reconnection interface, in accordance with an embodiment of the present invention.

a timeout or reconnection alert displayed through the user interface 134 on the display unit 104 of the user device 102, where the system 100 is notifying the user that the therapy session is entering an inactive state. The processing unit 124, through the session management module 126, is determining prolonged inactivity and preparing controlled disconnection procedures that maintain the integrity of experiential therapy data stored in the database 120. The user interface 134 shown in FIG. 5B is presenting options for immediate disconnection or rejoining the session, with each action being processed through the communication network 122 and directed to the application server 118. The synchronization control module 128 and the media stimulus delivery module 130 remain on standby as FIG. 5B handles user decision input through the keyboard 114, touch interface 112, or related components of the user device 102. All transitions shown in FIG. 5B are aligned with the secure storage operations performed through the data logging module 132 and the behavioral interpretation procedures handled through the smart computational artificial intelligence module 140.

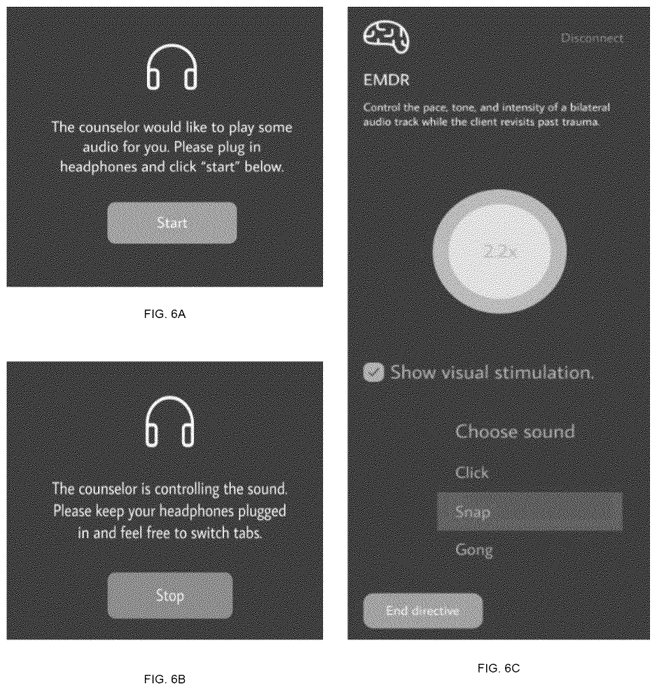

FIG. 6A illustrates an audio initiation interface for experiential therapy activation, in accordance with an embodiment of the present invention.

The audio initiation interface rendered on the display unit 104 of the user device 102 through the user interface 134. The user interface 134 is presenting an instruction that prepares the user to engage in bilateral auditory stimulation controlled through the processing unit 124 and the media stimulus delivery module 130. The display unit 104 is showing a structured communication prompt indicating that the counselor interface 136 is initiating an auditory phase of an experiential therapy module 142. The speaker unit 110 of the user device 102 is remaining in a ready state for reception of auditory stimulation instructions transmitted through the application server 118 and communication network 122. The synchronization control module 128 is actively aligning counselor-generated experiential commands with the corresponding audio event that the media stimulus delivery module 130 is distributing to the user. The interface presented in FIG. 6A demonstrates the start point where bilateral auditory instruction is transitioning from the counselor interface 136 to the user device 102 as part of the continuous experiential therapy process 200.

FIG. 6B illustrates an audio control interface managed by the counselor during an experiential session, in accordance with an embodiment of the present invention.

The moment during a live experiential therapy session where the counselor interface 136 is controlling auditory stimulation that the speaker unit 110 of the user device 102 is outputting. The user interface 134 displayed on the display unit 104 is informing the user that counselor-directed audio control is presently active and uninterrupted. Throughout this phase, the processing unit 124, operating through the synchronization control module 128, is translating continuous counselor instructions from the counselor interface 136 into real-time manipulation of auditory pacing, tone, and alternating left-right audio presentation. The communication network 122 is continuously transferring audio control commands and sensor-generated activity data to maintain precise bilateral stimulation timing. The media stimulus delivery module 130 is delivering auditory outputs while remaining functionally coordinated with real-time user behavior captured by the microphone 106, image capturing unit 108, touch interface 112, keyboard 114, and eye tracking sensor 116. FIG. 6B demonstrates how the system 100 maintains seamless server-driven control over sensory stimulus delivery during the therapy sequence.

FIG. 6C illustrates an experiential auditory-visual control interface for EMDR therapy, in accordance with an embodiment of the present invention.

The experiential control interface displayed on the display unit 104 of the user device 102, representing the counselor-oriented configuration of bilateral stimulation parameters accessed through the counselor interface 136. The user interface 134 presented in FIG. 6C is structured to show sound selection options and visual stimulation toggles that directly interface with the media stimulus delivery module 130 for real-time distribution of auditory and visual experiential stimuli. The processing unit 124 is continually receiving counselor selections and transferring these selections through the synchronization control module 128 to generate accurate experiential events on the user device 102. The database 120 and experiential therapy modules 142 stored within it are supplying the sound assets and visual patterns that the media stimulus delivery module 130 is outputting. The session management module 126 is maintaining the active state of the therapy session during these adjustments, while the data logging module 132 captures time-stamped information reflecting audio pacing, user reactions, and interaction changes. FIG. 6C therefore presents the structured control environment where auditory pacing, sound type, and optional visual stimulation parameters are aligned with bilateral experiential therapy delivery.

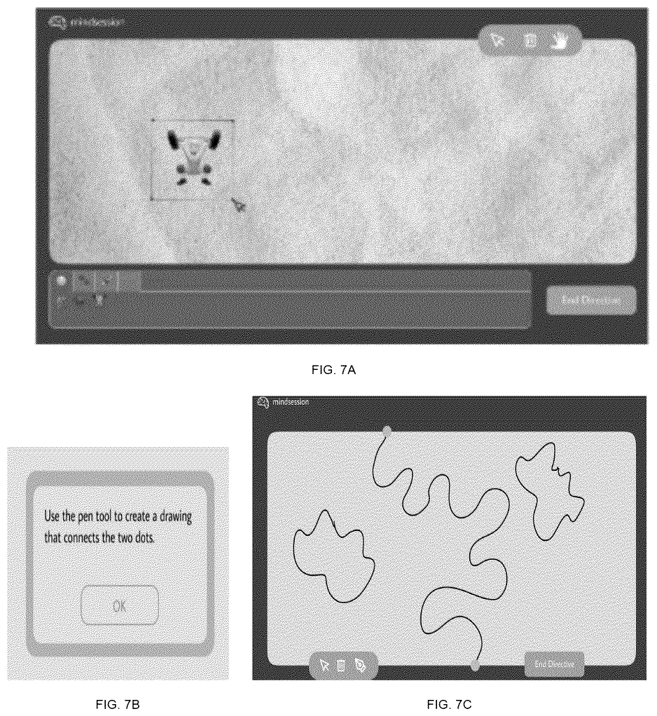

FIG. 7A illustrates an online sand tray experiential workspace rendered within a therapy session, in accordance with an embodiment of the present invention.

The online sand tray experiential interface being displayed on the display unit 104 of the user device 102 through the user interface 134 during a live therapy session within the system 100. The user interface 134 is presenting a textured sandbox surface on which expressive elements are being positioned and manipulated by the user through the touch interface 112. The image capturing unit 108 is continuously recording user expressions and posture changes as the experiential interaction proceeds, while the eye tracking sensor 116 is transferring eye movement information to the processing unit 124 through the communication network 122 for real time monitoring. Through the application server 118, the processing unit 124 is interpreting user movements and object placements using the synchronization control module 128 so that each action corresponds accurately to the counselor-driven commands originating from the counselor interface 136. The database 120 is supplying emojis, sticker assets, and object elements stored within the experiential therapy modules 142, enabling the media stimulus delivery module 130 to render them in the sand tray environment. The data logging module 132 is recording each drawing action, object movement, and interaction time stamp for long-term therapy session records. FIG. 7A shows the controlled expressive environment where tactile interaction, audiovisual feedback, and counselor coordination are functioning in a unified experiential framework.

FIG. 7B illustrates an instructional prompt shown to guide user interaction during an expressive experiential task, in accordance with an embodiment of the present invention.

The instruction window displayed on the display unit 104 of the user device 102 through the user interface 134, providing a directive to guide the user into an expressive drawing activity during process 200. The user interface 134 is rendering this prompt in conjunction with commands transmitted from the counselor interface 136 and interpreted through the synchronization control module 128 of the processing unit 124. The application server 118 is transferring the instructional data through the communication network 122 so that the user perceives the guidance without delay. The touch interface 112 of the user device 102 is being prepared to receive continuous drawing information as the user begins connecting the indicated points, while the data logging module 132 is prepared to record these interactions for later review. The display unit 104 is presenting the instructional layout in alignment with elements stored within the experiential therapy modules 142 housed in the database 120. FIG. 7B demonstrates how the system 100 delivers step-based experiential instruction that synchronizes seamlessly with expressive tasks governed by the processing unit 124.

FIG. 7C illustrates an experiential drawing interface generated during a collaborative or bilateral drawing directive, in accordance with an embodiment of the present invention.

a freeform drawing canvas shown on the display unit 104 of the user device 102 during a drawing-based experiential therapy activity executed within system 100. The user interface 134 is presenting a continuous stroke path generated through input captured by the touch interface 112 and the keyboard 114 directional controls. The synchronization control module 128 of the processing unit 124 is translating both counselor commands and user-generated drawing input into real time visual output that the media stimulus delivery module 130 is rendering on the display unit 104. The image capturing unit 108 and the microphone 106 are capturing contextual user responses and expressive reactions while the eye tracking sensor 116 is transmitting gaze information that supports evaluation of emotional focus during the activity. The database 120 is providing drawing parameters, canvas rules, and experiential structure stored within the experiential therapy modules 142 to maintain consistent session behavior. The data logging module 132 is recording line paths, drawing duration, movement intensity, and user interaction timing for integration into the overall session record. FIG. 7C depicts the environment where expressive motor actions, emotional cues, and real time counselor guidance are functioning as a coherent experiential sequence coordinated through the processing unit 124 and application server 118.

The best mode of operation of the online multifaceted therapy delivery system 100 and the process 200 begins with the activation of the user interface 134 on the display unit 104 of the user device 102, where the therapist initiates authentication by entering therapist credentials. These credentials are transferred through the communication network 122 to the application server 118, where the processing unit 124 verifies access through the session management module 126. After authentication, the processing unit 124 retrieves experiential therapy modules 142 and related experiential therapy activity scripts from the database 120 and displays them on the counselor interface 136 for selection. Once the therapist selects an experiential therapy module 142, the session link generation module 138 generates a unique session access link for transmission to the user device 102, activating the experiential therapy module and initiating live audiovisual communication.

During the therapy session, the user device 102 continuously captures microphone 106 information, image capturing unit 108 information, keyboard 114 inputs, touch interface 112 interactions, and eye tracking sensor 116 information. All captured information travels through the communication network 122 to the application server 118 and into the processing unit 124, allowing the synchronization control module 128 to produce corresponding experiential events that accurately reflect counselor instructions issued through the counselor interface 136. Simultaneously, the media stimulus delivery module 130 outputs bilateral auditory stimulation through the speaker unit 110 and renders visual stimulation on the display unit 104, which remains synchronized with therapist-driven experiential pacing.

The data logging module 132 continuously records sensor-generated information, audiovisual information, user interaction information, and experiential activity progression into the database 120, ensuring consistent session tracking. Throughout the session, the smart computational artificial intelligence module 140 analyzes user behavior patterns to generate experiential outcome information for real time review through the counselor interface 136. The system 100 maintains uninterrupted experiential interaction even when switching between multiple experiential therapy modules 142 within one session, preserving all experiential interaction states. At the end of the therapy session, the counselor interface 136 issues a termination command, which the session management module 126 executes, finalizing data storage and ensuring session closure within the database 120.

The online multifaceted therapy delivery system 100 is providing a tightly integrated technical platform that is improving fidelity of remote therapeutic interactions by coordinating sensory rendering, interaction capture, and session control in real time. The user device 102 is receiving multisensory experiential content and transmitting user responses while the display unit 104 is rendering interactive visuals and the speaker unit 110 is delivering synchronized audio. The application server 118 is orchestrating session flow and the processing unit 124 is executing experiential operations so that the communication network 122 is maintaining low latency bidirectional streams between the user device 102 and the application server 118. The user interface 134 is presenting task instructions and interaction surfaces while the counselor interface 136 is issuing live experiential commands that are being routed through the synchronization control module 128. The database 120 is storing module content and session logs and the data logging module 132 is preserving time stamped interaction records so that continuity and reproducibility of therapeutic work are being retained across sessions.

The system is offering technical advantages through several distinguishing technical aspects embodied across components that are functioning together. The session link generation module 138 is producing secure, session-specific access pathways that are being associated with experiential therapy modules 142 stored in the database 120, enabling on-demand activation without manual file sharing. The synchronization control module 128 is mapping counselor commands into immediate experiential events on the display unit 104 and the speaker unit 110 while the media stimulus delivery module 130 is producing coordinated bilateral audio and visual stimuli with precise timing. The smart computational artificial intelligence module 140 is continuously analyzing sensor-generated activity from the microphone 106, the image capturing unit 108, and the eye tracking sensor 116 to produce experiential outcome information that is being presented to the counselor interface 136 for interpretation. The processing unit 124 is supporting dynamic module switching without terminating audiovisual communication so that multiple experiential therapy modules 142 are being sequenced during a single uninterrupted session.

The present system 100 is addressing common limitations in distributed therapy setups by consolidating control, content, and telemetry into a single responsive environment where session management module 126 is validating access and enforcing session state while the synchronization control module 128 and the media stimulus delivery module 130 are preserving temporal alignment between stimulus and response. Fragmentation across separate tools is being replaced by an integrated flow where the database 120 is serving modular assets, the session link generation module 138 is enabling streamlined participant entry, and the data logging module 132 is creating auditable, time stamped records for therapeutic continuity. Behavioral signal processing through the smart computational artificial intelligence module 140 is enabling adaptive interpretation of engagement and experiential progression so that counselor decisions through the counselor interface 136 are being supported with synthesized outcome information. Together these components are producing a distinguishing therapeutic platform that is improving engagement, synchronization accuracy, and session reproducibility for complex experiential interventions.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

1. An online multifaceted therapy delivery system and process thereof, the system comprises:
a. A persistent storage system, to store:
i. A library of metadata (e.g. name, description) about therapeutic experientials.
ii. Connection information (e.g. links) for specific sessions of therapeutic experientials.
iii. Client and counselor data collected during execution of therapeutic experientials.
iv. Associations between each counselor and an active session.
v. Authentication information for counselors.
b. An application server that does the following:
i. Provide functionality for the counselor device (see below) to authenticate itself.
ii. Provide functionality for the counselor device to create a new “session” of a particular experiential. In this case, the server creates and stores connection information for the experiential before returning this connection information to the counselor device.
iii. Provide functionality for the counselor device to retrieve the active session for the counselor and its connection information.
iv. Provide functionality for the counselor and client devices (see below) to retrieve and modify data of an active session that they have the connection information for.
v. Provide functionality to proactively send data to counselor and client devices when a session they are connected to is modified.
vi. Provide functionality for the counselor device to terminate their active session and provide for whatever data archival or deletion is required to comply with pertinent healthcare data law (e.g. HIPAA).
vii. Provide functionality for the client and counselor devices to retrieve a script that executes the primary business and display logic of an experiential given connection information.
c. A client device (e.g. a smartphone or laptop) comprising:
i. A display unit, to visually render experiential therapy content during the live therapy session.
ii. At least one of:
1. A touch interface.
2. A keyboard.
3. A mouse.
iii. A processor, to run experiential scripts provided by the application server.
iv. A means of connecting to the application server (e.g. an internet connection).
d. A counselor device (e.g. a smartphone or laptop) comprising:
i. A display unit, to visually render experiential therapy content during the live therapy session.
ii. At least one of:
1. A touch interface.
2. A keyboard.
3. A mouse.
iii. A processor, to run experiential scripts provided by the application server.
iv. A means of connecting to the application server (e.g. an internet connection).
e. The following software modules:
i. A “data synchronization” module that runs on the client and counselor devices. This module is responsible for:
1. Storing session data on the counselor and client devices in a way that experiential scripts can access and manipulate it.
2. Sending and receiving data to/from the application server in order to keep the copy on the client device in sync with the copy on the counselor device.
ii. A “session management” module that is responsible for managing an individual execution of a therapeutic directive.
iii. A library of “experiential” modules that are responsible for executing the business and display logic of each therapeutic directive.

2. The system of claim 1, wherein the wherein the counselor and client devices further comprising a speaker and experiential modules are modified to support conveying auditory information via this speaker.

3. The system of claim 1, wherein the client device supports eye-movement tracking and experiential modules are modified to support collecting and using this data.

4. The system of claim 1, wherein the further comprising a counselor interface connected to the synchronization control module through the application interface and configured to allow the counselor to issue real time experiential instructions that generate experiential events on the user device.

5. The system of claim 1, wherein the system further comprising a smart computational artificial intelligence module configured to analyze sensor-generated activity and experiential interaction patterns and to generate a summarized experiential profile for the counselor to review.

6. The system of claim 1, wherein the system further comprising an additional server component to relay an audiovisual connection between the counselor and client device.

7. The system of claim 1, wherein the communication network is further configured to transfer multi-participant audiovisual information, allowing multiple users to join the therapy session simultaneously under the control of the counselor.

8. The system of claim 1, wherein the synchronization and session modules are further configured to switch between multiple experiential therapy modules during one uninterrupted therapy session without terminating the session.

9. The system of claim 1, wherein the display module is further configured to deliver bilateral visual stimulation and bilateral auditory stimulation in response to commands received from the counselor interface.

10. A process for online multifaceted therapy delivery, the process comprising:
a. receiving therapist authentication credentials through a user interface and transferring the therapist authentication credentials through a communication network to a processing unit of an application server for verification through a session management module of the processing unit;
b. retrieving a library of experiential therapy modules from a database connected to the application server and displaying the library of experiential therapy modules on a counselor interface connected to the synchronization control module of the processing unit to enable therapist selection of an experiential therapy module;
c. generating a unique session access link through a session link generation module of the processing unit and transmitting the unique session access link to a user device through the communication network for activation of the experiential therapy module during a live therapy session;
d. establishing audiovisual communication by receiving microphone information, visual information from an image capturing unit, keyboard inputs, touch interaction information, and eye movement information from the user device and transmitting the received information through the communication network to the processing unit of the application server for execution of the experiential therapy operations;
e. rendering experiential therapy content by delivering visual stimulation on a display unit of the user device and delivering auditory stimulation through a speaker unit of the user device through a media stimulus delivery module of the processing unit in response to live experiential commands originating from the counselor interface;
f. generating synchronized experiential events by mapping each experiential command from the counselor interface to a corresponding experiential activity on the user device through the synchronization control module of the processing unit during the therapy session;
g. executing a continuous experiential therapy loop by receiving real time user interaction information from the touch interface, the keyboard, the microphone, the image capturing unit, and the eye tracking sensor and transmitting the real time user interaction information to the processing unit for live modification of experiential stimuli through the synchronization control module;
h. terminating the therapy session by receiving a termination command from the counselor interface and executing session closure through the session management module for final storage of therapy session information in the database;

11. The process of claim 10, wherein the process further comprising sequentially switching between multiple experiential therapy modules during a single therapy session without exiting the therapy session and without terminating audiovisual communication.

12. The process of claim 10, wherein the process further comprising using an artificial intelligence module to interpret client data, the output of which is provided to the counselor for display and analysis.

13. The process of claim 10, wherein the process further comprising displaying counselor driven experiential instructions on the user interface of the user device and receiving user expressive responses through the user interface for transmission to the processing unit for real time therapy progression control.

14. The process of claim 10, wherein the process further comprising receiving multi-participant audiovisual information from two or more user devices through the communication network and synchronizing the experiential activities across multiple user devices under the control of the counselor interface during the live therapy session.

15. The process of claim 10, wherein the process further comprising executing an experiential therapy activity that involves interactive drawing across multiple users by receiving sequential line-drawing interaction information from multiple user devices and merging the line-drawing interaction information into a single shared experiential drawing output through the synchronization control module.

16. The process of claim 10, wherein the process further comprising delivering bilateral stimulation during the experiential therapy content by transmitting alternating visual stimulation and alternating auditory stimulation to the display unit and the speaker unit in direct response to bilateral stimulation instructions issued through the counselor interface

17. The process of claim 10, wherein the process further comprising receiving selection of expressive activity assets from a graphical asset library stored in the database and rendering the expressive activity assets in an object manipulation workspace on the display unit of the user device for experiential therapy involvement.

18. The process of claim 10, wherein the process further comprising generating an experiential mapping display that includes labeled imagery provided by the client and transmitting the labeled imagery mapping display to the counselor interface for real time review during the live therapy session.

19. The process of claim 10, wherein the process further comprising executing a mindfulness experiential activity by receiving sound identification responses from the user through the microphone and the keyboard and transmitting the sound identification responses to the processing unit for validation through the smart computational artificial intelligence module.

20. The process of claim 10, wherein the process further comprising receiving continuous bilateral drawing activity information from the touch interface and from keyboard directional keys of the user device and converting the bilateral drawing activity information into an abstract experiential drawing display through the media stimulus delivery module.
